(19) United States
(12) Patent Application Publication
(10) Pub. No.: US 2023/0282338 A1
(43) Pub. Date: Sep. 7, 2023

(54) SYSTEMS AND PROCESSES FOR DELIVERING PROCEDURAL CARE OPTIONS, INCLUDING SPECIALIZED PROGRAMMING AND OPTIMIZED WEB INTERFACES

(71) Applicant: Stephanie Haskell Rees, Redondo Beach, CA (US)

(72) Inventor: Stephanie Haskell Rees, Redondo Beach, CA (US)

(21) Appl. No.: 17/962,128

(22) Filed: Oct. 7, 2022

Related U.S. Application Data
(60) Provisional application No. 63/253,642, filed on Oct. 8, 2021.

(51) Int. Cl. G16H 40/20; G16H 10/60; G16H 50/70; G16H 70/20

(52) U.S. Cl. CPC G16H 40/20; G16H 10/60; G16H 50/70; G16H 70/20; G16H 10/20

(57) ABSTRACT

FINDR-band of process and system provides insight and understanding to body mechanics and dynamics, while using technology-based algorithms to deliver three dimensional models of procedural care options, as a web-based tool using evidence and peer reviewed data.

### CROSS-REFERENCE TO FUNDING SOURCES & RELATED CASES

No government funding was used in connection with the instant provisional patent application and its filing, which claims priority of U.S. provisional Ser. No. 63/253,643 filed 7 Oct. 2021.

### BACKGROUND OF THE INVENTIONS

The present disclosures relate to connecting patients to the most appropriate phy-sicians/doctors/licensed care providers. In specific, FINDR™ restores confidence in use of data, medical terminology and tailored algorithms to navigate through the ostensive maze of medical malapropisms, sponsored ads and accreditations, which do not provide insight into the listed caregivers. Longstanding needs are addressed this way, including a prime and species-esque use case for ortho and spine issues as set further herein and claimed below, for example in FIG. 1. The instant inventions impact second episodes by reduction or elimination of the same, which constitutes progress in science and the useful art qualifying for US Letters Patent, it is respectfully proposed, as shown below and within the scope of the claimed subject matters offered for consideration.

### OBJECTS AND SUMMARIES OF THE INVENTIONS

Briefly stated, the instant inventions (styled as FINDR™/FINDR herein, definitionally, trademark rights pending also, are tools designed to link patients' medical needs with the care provider most likely to support their needs in light of objective data, and certified peer reviewed inputs) provide insight and understanding to body mechanics and dynamics, while using technology-based algorithms to deliver three dimensional models of procedural care options via enhanced and improved interfaces with the web. Likewise, the imnstasnt inventions contemplate at least one of these steps being supplanted by a questionnaire administered with the instant system and processes, as shown in exemplary, but not limiting detail below.

According to embodiments, there are provided systems to retrieve the ideal doctor, for example, for surgical needs, based upon patient profiles modified by information about patients in terms of adaptive and selected programming and proprietary algorithms.

According to embodiments, there is disclosed a system which functions by the steps of generating, providing profiles and backgrounds, and ranking potential care providers.

According to embodiments, systems use programmed outcomes driven by objective and data-backed criteria delivered seamlessly through the subject web interface.

According to embodiments, medically trained professions are used both as targets and to confirm objective data-points and suggested outcomes, including for example surgeons, other healthcare providers and interventionalists, cardiologist, vascular surgeons and ad-visors from physical therapists to psychologists.

According to embodiments, unsuccessful outcomes are mitigated or avoided by the FINDR verified process, optimized in the fully verged and described attributes of FINDER FLOW defined in the claims below, shown in the fugues appended hereto and manifested in the intellectual property making said subject matter eligible for protection of US Letters Patents, into alia.

Various objects, features and aspects of the inventive subject matter shall become more apparent from the following detailed descriptions of the preferred embodiments, and those fully enabled species of the inventive genus, wherein as with the accompanying drawing figures, schematics and cartoons in which like numerals represent like components.

### BRIEF DESCRIPTION OF THE FIGURES

The above-mentioned features are illustrated along with plethoric objects embodied by and implemented according to those known web-based protocols, in addition to interfacing with operating systems, Javascript™ brand or other API, as would be operable and functional to those of skill in the art, or implemented as fair use pursuant to Google v. Oracle (citation omitted), meaning that the instant platform as configured is both enabled and practical for this interface as optimized, but ostensively re-configurable as needed, wherein:

FIG. 1 is a schematic cartooned set of exemplary screen shots showing, for example, the subject use case embodied in the three tranche FINDR VERIFIED PROCESS, as applied to in this illustration ortho, spinal specialties;

FIG. 2A is a schematic cartooned set of screen shots continuing the narrative of work flow according to the instant system and to the teachings of the present inventions; and

FIG. 2B continues to illustrate workings of the instant inventions using cartooned screen shots depicting a user's perspective while working with the instant process and systems;

FIG. 2C similarly illustrates the work flow on the way to the FINDR Verified Match;

FIG. 3 likewise illustrates an end-point summary according to the teachings of the instant disclosure, in identical schematic cartooned screen shot fashion;

FIG. 4 is a series of three flow charts which completes the summary depictions by showing how the FINDR experience, quality overlay and Verified Match render both the systems and process readily accessible and primed to improve on the second episodes conundrum, which plagues the prior art and extent systems.

FIG. 4A shows the first of a series of three flow charts demonstrating the process of the present invention;

FIG. 4B shows the second of a series of three flow charts illustrating five process steps according to the workings of the present invention; and

FIG. 4C completes the three flow chart steps by setting forth the final steps according to the process of the present invention.

To the extent needed, corresponding reference characters indicate corresponding components throughout the several views of the drawings. Screen shots shown of the resultory web-based system are shown to demonstrate how they work, artisans understanding that key features are representative of desiderata mapped from customer facing patient needs, in light of data acquired, and specialty addressed, meaning that by showing north and spinal application but a straight forward example of there larger fields to which the present invention applies is projected to owe within the claims of the subject matters patentable herein, inter alia. Skilled artisans will appreciate that elements in the figures are illustrated for simplicity and clarity, and have not necessarily been drawn to scale. For example, the dimensions of some of the elements in the figures may be exaggerated relative to other elements to help to improve understanding of various embodiments of the present invention. Also, common but well-understood elements that are useful or necessary in a commercially feasible embodiment are often not depicted in order to facilitate a less obstructed view of these various embodiments of the present invention.

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary, and that the actual parameters, dimensions, materials, and/or configurations will depend upon the specific application or applications for which the teachings of the present disclosure is/are used.

### DETAILED DESCRIPTION OF PREFERRED EMBODIMENTS

The present inventor has defined a set of problems and crafted solutions for the same, using both proprietary and public domain aspects to create a universal health care solution, delivered on the web and supporting numerous interfaces managed by its algorithm set, along with data refined back into the system. Reduction and elimination of second episodes becomes clearly advantageous when searching for medical solutions of immense magnitude and critical temporal importance. By establishing the first true mechanism to educate patients, while being able to separate and certify the best specialists, the instant inventions have the potential to restore confidence in the health care access and systems which exist and shall be developed.

By offering for consideration novel enhanced systems to generate perfect surgeon matches based upon patients'/users' needs and ranked by preferences, progress in science and the useful arts has been achieved, worthy of United States Letters Patent, as detailed and claimed below.

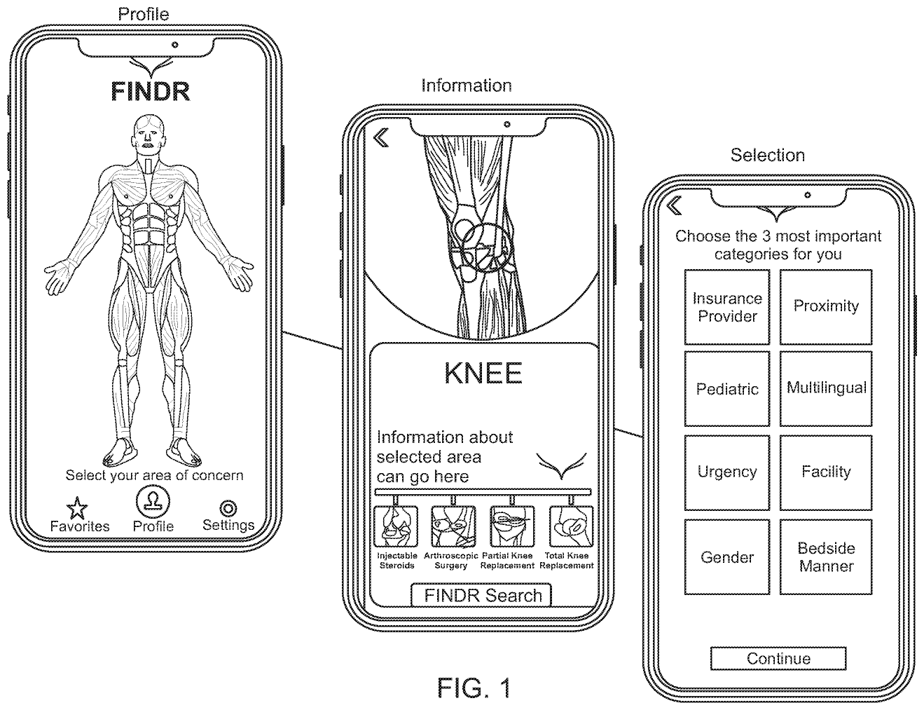

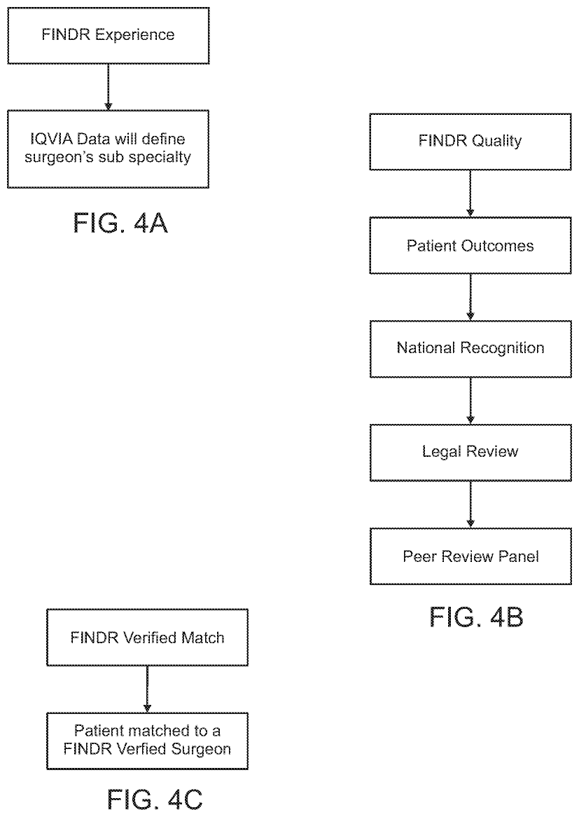

Referring now to FIG. 1, it is manifest that at least a minimum number of exemplary screen capture views to illustrate tenets of the instant system. Those of skill in the art understand this Figure (and the others) to explain and verify the use case of ortho and spinal as illustrative but not limiting to the instant teachings. As with all of the schematics herein, FIG. 1 offers for consideration a visible and transparent route to the three tenets of the present inventions (see FIGS. 4A, B and C) the unique FINDR Experience, FINDR Quality and FINDR Verified Match. FIG. 1's first screen shot shows the initial Profile building steps, namely the data which is required to fortify the section process but does not impact HIPAA or privacy issues. It is also understood that patient and user questionnaires may collect or verify the information n gathered. For the purpose of this use case the Profile gathers infraction relative to the KNEE, where the second level or FINDR Quality section can filter and use machine learning or AI (moderated by the system in this case) to deliver and administer all of the way through the Peer Review Panel and FINDR Verified Match (see FIG. 4B). Selection continues the information and data harvesting elements offered for consideration, which are arrayed to allow them to be vetted by the algorithms against the criteria generated.

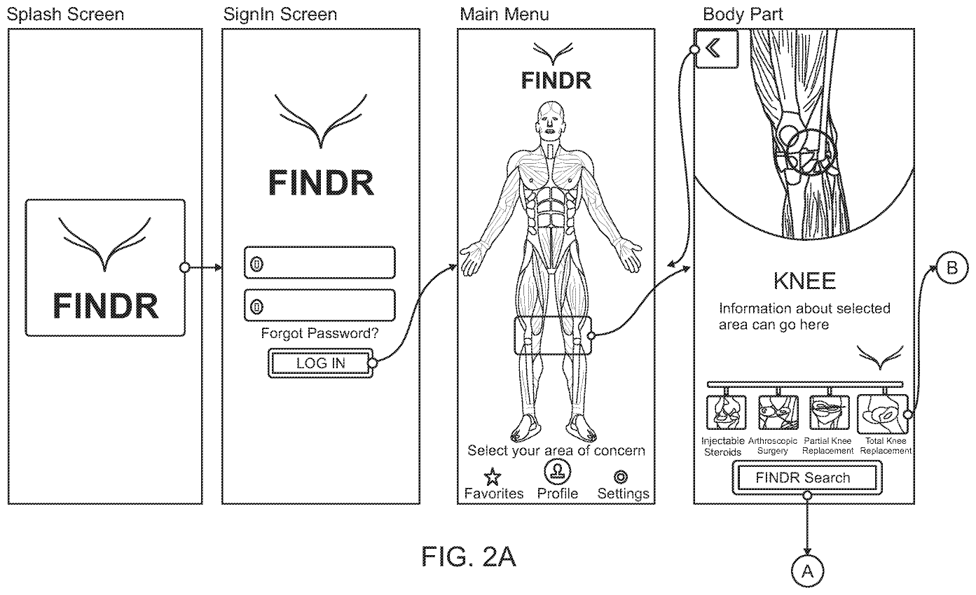

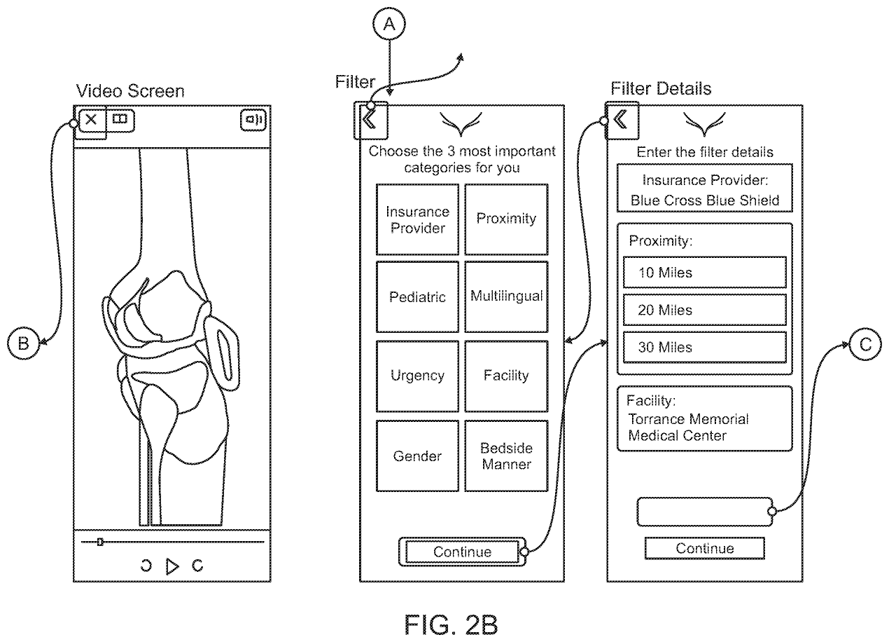

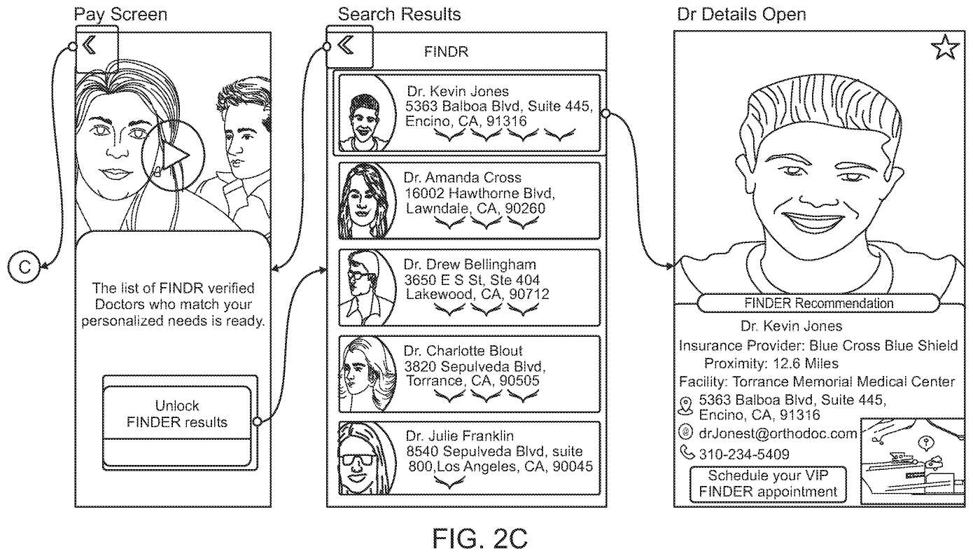

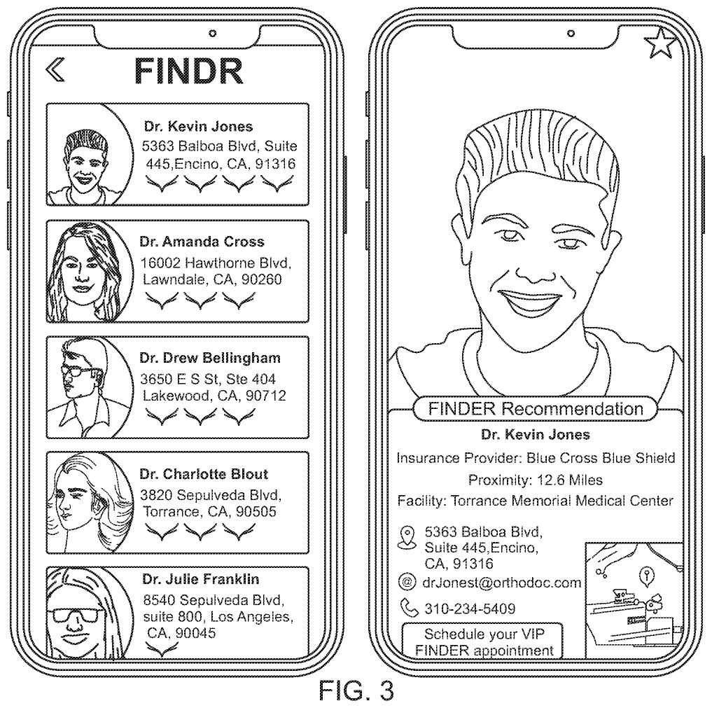

Referring now also to FIG. 2A, 2B, 2C and to FIG. 3, workflow of exemplary embodiments of the present invention are shown in illustrative but not limiting form. For example, from the data structures used to array user data for the algorithms herein we offer user friendly splash screens, sign-in screens and main page interface which allows users to facilely navigate to and update their profiles. The main menu supported both the profile generation and selection of an area of the body to be addressed. FIG. 2A opens with a splash screen depicting the FINDR LOGO (aka FINDR & DESIGN at the USPTO), which denotes the alignment of a user's selection for example, by dynamically being arrayed next to the item selected. Moving through the splash screen for example conventional login parameter may be used or meta-data encrypted passwords. Note the FINDR LOGO showing that choices or data input elements selected have been accepted. Treatment options are likewise proffered as the area shown (KNEE), and herein the choice of Total Knee Replacement B has directed the FINDR Search as shown by the FINDER logo element hovering over choice B. FIG. 2B likewise progresses from A to B to C on the basis of input and the filters data set regarding care providers, inter alia. FIG. 2C includes the payment screen and C showing how to navigate thereto. By continuing to FIG. 3, an exemplary but not limiting data set is shown which offers for consideration a number of service providers from whom is created a resultory FINDER Certified Recommendation, an contemporaneous support with VIP Appointment setting.

Mindful that more than 1 million elective procedures are performed annually in the United States, at least 850,000 cases comprise hip, knee and spinal procedures. The body part screen allows users to zoom in on complex anatomical structures to identify needs for treatment, pinpoint details of the same, and to undertake a FinDr™ type of search. By inputting insurance data, proximity, and facility fields, in light of the body part chosen inputs connect to the algorithm set and continue to generate options via the Filter and Filter Detail screens.

A pay screen (FIG. 2C at letter C) for example, which then allows a list of results to be arrayed once the user-friendly instructions are followed. Search results follow, typically as shown giving 5-6 options along with the ability to scroll through and select details on each selection.

FIGS. 4A, 4B and 4C each likewise shows the results of use of the proprietary algorithms and details on recommendations. Once a user chooses a doctor, then an appointment is created. Searches are saved and may be accessed, revised, and repeated. The FINDR Experience involves database and databank creation by specialty, likewise mining of IOVIA types of data further define a surgeon's experience in a sub specialty. FINDR Quality means that Patient Outcomes and National recognition of the validity of the data, incorporated with Legal Review and the Peer Review Panel is done to validate the FINDR Verified Match, wherein each Patient, for example is matched to a FINDR Verified Surgeon.

Artisans clearly understand that the instant system creates an opportunity to cross into all specialties from cardiovascular to neurovascular, bridging the gap from elective to required treatments.

Moving forward, patient and user confidence can be accelerated and it is respectfully proposed, positively enhanced, by the instant teachings.

Those skilled in the art will recognize, or be able to ascertain using no more than routine experimentation, many equivalents to the specific embodiments of the disclosure described herein. It is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

All definitions, as defined and used herein, should be understood to control over dictionary definitions, definitions in documents incorporated by reference, and/or ordinary mean-ings of the defined terms.

The indefinite articles “a” and “an,” as used herein in the specification and in the claims, unless clearly indicated to the contrary, should be understood to mean “at least one.” The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified, unless clearly indicated to the contrary.

Reference throughout this specification to “one embodiment” or “an embodiment,” means that a particular feature, structure, or characteristic described in connection with the embodiment is included in at least one embodiment. Thus, appearances of the phrases “in one embodiment”, or “in an embodiment” in various places throughout this specification are not necessarily all referring to the same embodiment. Furthermore, the particular features, structures, or characteristics may be combined in any suitable manner in one or more embodiments.

The terms and expressions which have been employed herein are used as terms of description and not of limitation, and there is no intention, in the use of such terms and expressions, of excluding any equivalents of the features shown and described (or portions thereof), and it is recognized that various modifications are possible within the scope of the claims. Accordingly, the claims are intended to cover all such equivalents.

Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar throughout this specification may, but do not necessarily, all refer to the same embodiment.

Furthermore, the described features, structures, or characteristics of the invention may be combined in any suitable manner in one or more embodiments. In the following description, numerous specific details are provided to provide a thorough understanding of embodiments of the invention. One skilled in the relevant art will recognize, however, that the invention may be practiced without one or more of the specific details, or with other methods, components, materials, and so forth. In other instances, well-known structures, materials, and so forth. In other instances, well-known structures, materials, or operations are not shown or described in detail to avoid obscuring aspects of the invention.

The schematic flow chart diagrams included herein are generally set forth as logical flow chart diagrams. As such, the depicted order and labeled. steps are indicative of one embodiment of the presented method. Other steps and methods may be conceived that are equivalent 111 function, logic, or effect to one or more steps, or portions thereof, of the illustrated method. Additionally, the format and symbols employed are provided to explain the logical steps of the method and are understood not to limit the scope of the method. Although various arrow types and line types may be employed in the flow chart diagrams, they are understood not to limit the scope of the corresponding method. Indeed—some arrows or other connectors may be used to indicate only the logical flow of the method. For instance, an arrow may indicate a waiting or monitoring period of unspecified duration between enumerated steps of the depicted method. Additionally, the order in which a particular method occurs may or may not strictly ad-here to the order of the corresponding steps shown.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Specific embodiments disclosed herein may be further limited in the claims using, consisting of, or consisting essentially of language. When used in the claims, whether as filed or added per amendment, the transition term “consisting of” excludes any element, step, or ingredient not specified in the claims. The transition term “consisting of” essentially limits the scope of a claim to the specified materials or steps and those that do not materially affect the basic and novel characteristic(s). Embodiments of the invention so claimed are inherently or expressly described and enabled herein.

As one skilled in the art would recognize as necessary or best-suited for performance of the methods of the invention, a computer system or machines of the invention include one or more processors (e.g., a central processing unit (CPU) a graphics processing unit (GPU) or both), a main memory and a static memory, which communicate with each other via a bus.

A processor may be provided by one or more processors including, for example, one or more of a single core or multi-core processor (e.g., AMD Phenom II X2, Intel Core Duo, AMD Phenom II X4, Intel Core i5, Intel Core I & Extreme Edition 980X, or Intel Xeon E7-2820).

An I/O interface may include a video display unit (e.g., a liquid crystal display (LCD) or a cathode ray tube (CRT)), an alphanumeric input device (e.g., a keyboard), a cursor control device (e.g., a mouse), a disk drive unit, a signal generation device (e.g., a speaker), an accelerometer, a microphone, a cellular radio frequency antennae, and a network interface device (e.g., a network interface card (NIC), Wi-Fi card, cellular modem, data jack, Ethernet port, modem Jack, HDMI port, mini-HDMI port, USB port), touchscreen (e.g., CRT, LCD, LED, AMOLED, Super AMOLED), pointing device, trackpad light (e.g., LED), light/image projection device, or a combination thereof.

Memory according to the invention refers to a non-transitory memory, which is provided by one or more tangible devices which preferably include one or more machine readable medium on which is stored one or more sets of instructions (e.g., software) embodying any one or more of the methodologies or functions described herein. The software may also reside, completely or at least partially, within the main memory processor, or both during execution thereof by a computer within system, the main memory and the processor also constituting machine-readable media. The software may further be transmitted or received over a network via the network interface device.

While the machine-readable medium can in an exemplary embodiment be a single medium, the term “machine-readable medium” should be taken to include a single medium or multiple media (e.g., a centralized or distributed database, and/or associated caches and servers) that store the one or more sets of instructions. The term “machine-readable medium” shall also be taken to include any medium that is capable of storing, encoding or carrying a set of instructions for execution by the machine and that cause the machine to perform any one or more of the methodologies of the present invention. Memory may be, for example, one or more of a hard disk drive, solid state drive (SSD), an optical disc, flash memory, zip disk, tape drive, “cloud” storage location, or a combination thereof. In certain embodiments, a device of the it1vemion includes a tangible, non-transitory computer readable medium for memory. Exemplary devices for use as memory include semiconductor memory devices, (e.g., EPROM, EEPROM, solid state drive (SSD), and flash memory devices, (e.g., SD, micro SD, SDXC, SDIO, SDHC cards); magnetic disks, (e.g., internal hard disks or removable disks); and optical disks (e.g., CD and DVD disks).

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

What is claimed is:

1. In a web-based system for optimized harvesting, packaging and offering for consideration to patients heath care provider data, improvements comprising, in combination:
profile generation;
therapy needs mapping; and
dynamically arraying care provider choices, with a Peer-reviewed Physician management network and system to provide a verified match.

2. The web-based system of claim 1, further comprising:
delivering three-dimensional tours of procedural care options, which comprises, in combination:
generating a profile for a patient including disclosed medical data and ostensive needs/desiderata;
providing background information regarding procedures at issue; and,
ranking potential care providers via professionally peer-reviewed, audited and computer-implemented referral system.

3. The web-based system of claim 2, further comprising at least an algorithm set based upon body mechanics and conditions, preferred care providers (for example) surgical metrics and reproducible outcomes, with optional AI and required human cross-referencing.

4. The web-based system of claim 3, whereby, a user is educated upon a full panoply of surgical options, offered for consideration post qualified professional peer reviewed oversight.

5. The web-based system of claim 4, including a sub-system for providing care giver/surgeon by FINDR Verified referral, comprising, in combination:
at least an unbiased, peer reviewed and electronically vetted product,
whereby subject care givers/surgeons are ranked based upon high-volume, low complications, ethical evaluations and specialized quality of care.

6. Improved systems using objective criteria to avoid unsuccessful outcomes by educating users on all surgical options, narrowing choices based upon physical needs and personal preferences embodied in FINDR™ generated profiles.

7. Improved systems of claim 6, further comprising peer reviewed referral networks having
at least a surgical panel member per medical specialty.

8. Improved systems of claim 7, further comprising visual learning tools.

9. Improved systems of claim 8, being a one-stop-shops for finding a care provider/surgeon.

10. Improved systems of claim 9, further comprising a trustworthy brand, such as FINDR™ enabling instant access with associated application technology.

11. A process to reduce or eliminate second episodes based upon supplying potential patients with data-driven recommendations, further comprising: care giver/surgeon, facility, and insurance company buy-in.

12. The process of claim 11, comprising, in combination:
a FINDR Experience, meaning IQVIA data to define care giver/surgeon's experience in a subspecialty based on value of case load.

13. The process of claim 12, further comprising:
FINDR Quality, meaning Patient Outcomes; National Recognition; Legal Review and at least a peer reviewed panel.

13. The process of claim 14, further comprising: at least a FINDR Verified Match, meaning a patient matched to a FINDR verified care giver/surgeon.

14. The process of claim 13, further comprising, in combination:
at least one specialty selected from the group of orthopedics and spinal management.

15. The process of claim 14, whereby more economically efficient outcomes are saved, arrayed and sorted using electronic storage means, including AI filters mediated by humans.

16. Products, by the process of claim 15, empowering users to avoid>320,000 mortal complication events annually, using data-driven products, coupled with physician referral supplements.

17. A process for designing a novel enhanced application and supporting architecture for a tri-partate approach to finding the best doctors, which comprises:
identifying an area of the body in three dimensions;
understanding surgical options with three-dimensional videos of surgical timelines for minimally invasive to open surgery; and,
completing via doctor/physician/surgeon referral for each users' preferences and needs.

18. The process of claim 17, driven by total procedural volume and number of surgeons performing surgeries in the United States.

19. The process of claim 18, being a web-based tool starting the FINDR three pronged approach to generate a profile; harvest information about patient needs in terms of alignment with best patient outcomes for areas of concern; and proffer recommendations for FINDR Verified Matches of care givers/surgeons associated by said data with hip, knee, and spinal procedures;
Wherein said data sets are fed back into the system with AI filters mediated by humans to cause continual updating.

20. The processes of claim 19, including other procedures ranging from cosmesis to cardiovascular and neurovascular procedures and causes.
